(19) United States
(12) Patent Application Publication
(10) Pub. No.: US 2011/0155294 A1
(43) Pub. Date: Jun. 30, 2011

(54) Method to Bond Materials using Surface Bonds with UV Catalysis and Composite Optical- and Electro-Optical Devices

(76) Inventor: Kevin Cyrus Robinson, Lutz, FL (US)

(21) Appl. No.: 12/649,986

(22) Filed: Dec. 30, 2009

(51) Int. Cl. B32B 37/00; C08F 2/48; C07C 1/00

(52) U.S. Cl. 156/60; 427/508; 204/157.15

(57) ABSTRACT

A method of forming permanent bonds is disclosed. The method allows components with in-situ surface hydroxyl bonds to be approximated, then illuminated with ultra-violet (UV) radiation to permit bonding, as well as filling an uneven or porous surface with a UV-catalyzing mixture to reduce infiltration and enable surface generation of radicals. The method envisions illuminating a photocatalyst material placed on one or both surfaces to create hydroxide (OH⁻) and superoxide anion (O2⁻) or other radicals, from in-situ H₂O or other species including organics, at a low enough temperature to prevent phase changes and interdiffusion between bonded species; these radicals network bond both with the surfaces and surrounding complexes. Nanometer-sized complexes are transparent, with large surface areas and allow for effective chemical catalysis. We disclose applications; also, that pure highly distilled H₂O can be used for bonding under certain conditions, as can acids such as hydrofluoric acid, with or without UV-catalysis.

### BACKGROUND OF THE INVENTION

Surface bonding is useful in many fields. For example, many high-performance optical components require robust, laser damage-resistant permanent bonding for assembly into composite devices. This has been attempted using organic cements, such as epoxies, but these generally do not stand up well to high powered laser illumination, yielding at lower energies, perhaps in the range of about 1 j/cm̂2 at a wavelength of 1064 nm. Note that many modem systems require laser damage thresholds in excess. of 5 j/cm̂2 at a wavelength of 1064 nm.

In other cases, surface bonding of photocatalyst particles has been attempted through the use of organic, or organic-inorganic combinations of, binders. This approach has the disadvantage of using binders; these are often less suitable, less durable materials than the underlying substrate and are typically partly or wholly incompatible with the latter.

### SUMMARY OF THE INVENTION

The disclosed approach uses inorganic bonding methods and processes for greater robustness to laser illumination and other environmental stresses. Inorganic bonding has the benefit of being compatible with inorganic substrates and being more mechanically durable. The method bonds UV-catalysts to the surfaces, and the surfaces to each other, by generating hydroxyl bonds and through physisorption.

### BRIEF DESCRIPTION OF DRAWINGS

The following drawings describe the bonding process, illustrating claim 3.

FIG. 1: SUBSTRATE—NEAR-SURFACE, CUT VIEW

1. Substrate, before treatment

2. Bulk material zone

3. Near-surface zone with voids and pores

FIG. 2: SUBSTRATE—NEAR-SURFACE, CUT VIEW

1. Substrate,, during treatment

2. Bulk material zone

3. Reactant mixture soaking into and reacting with near-surface zone, percolating through voids and pores

FIG. 3: SUBSTRATE—NEAR-SURFACE, CUT VIEW

1. Substrate, after dehydration

2. Bulk material zone

3. Treated, strengthened near-surface zone with reduced permeability due to reaction products

4. Thin chemically and mechanically resistant film of reaction products, excess reactants and physisorbed photocatalyst; covers surface, voids and pores

### DETAILED DESCRIPTION OF THE INVENTION

A method of forming permanent bonds, and devices assembled by this method, are disclosed. The disclosed method and process allow similar or dissimilar crystalline, vitreous or polycrystalline ceramic, metallic or organic polymeric components with in-situ surface hydroxyl bonds, such as optical flats, to be first joined by optical contacting or other close approximation and then illuminated with ultra-violet (UV) radiation to complete the bonding process. We also disclose that pure highly distilled H2O, as an amphoteric, provides some hydroxide and hydrogen ions at room temperature and so can be used for bonding under certain conditions, as can acids such as hydrofluoric acid; these may serve as adjuncts to the UV bonding method. These methods were first disclosed in Provisional Patent Application No. 61/200,920, filed Dec. 5, 2008.

The disclosed UV bonding envisions a coating of complexes of material such as nanoscale photoactive titania (TiO2) placed on one or both surfaces to UV-catalyze the creation of additional hydroxide (OH) and superoxide anion (O2-) or other radicals from in-situ H2O or other species, including organics. These radicals then network with the surfaces and surrounding complexes to generate reaction sites and so expedite bonding. Bonding may occur in many materials, including but not limited to silicic materials such as fused silica, optical glasses, ceramics, concrete, or other oxides and crystals. These materials may also harbor some surface organics that decompose under UV-illumination and generate additional radicals.

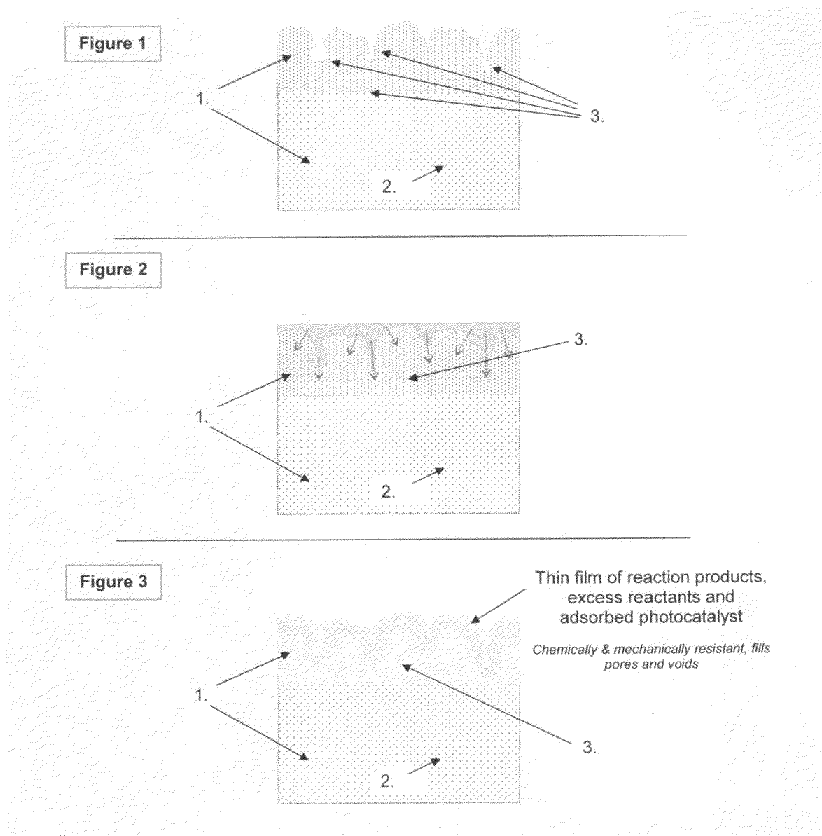

The method also admits filling an uneven or porous surface with a type of UV-catalyzing reactive mixture to reduce infiltration and enable surface generation of radicals, as shown in exemplary FIGS. 1 to 3. FIG. 2, section 3 shows how the disclosed mixture penetrates and reacts, while FIG. 3, section 3 illustrates how the reaction itself leaves a less-permeable, strengthened, photoactive near-surface zone, without using a binder.

The use of nanometer-sized complexes has the benefit of being transparent to optical wavelengths, while having large surface areas for more-effective chemical catalysis. We disclose that UV-catalyzed bonding can be performed at a low enough temperature to prevent phase changes and interdiffusion between bonded species. Stable bonds can be formed between materials of widely differing physical, mechanical, thermal, optical and electro-optical properties such as different hardness, chemical durability, chemical doping, mechanical strength, coefficients of thermal expansion, thermal conductivity, crystal structure, refractive indices, optical birefringence, nonlinear optical coefficients, electrical conductivity or semiconducting properties using UV illumination.

Exemplary bonding of two materials through UV-catalyzed networks is achieved at room temperature by applying a colloid of nanometer sized titania to at least one of the two bonding surfaces and placing the surfaces sufficiently close to each other to form a bridging chemical bond between them. Note that in the case of UV-opaque materials, the exemplary catalytic coating can first be illuminated in order to generate the radicals and then approximating the surfaces for bonding; also, materials that are ostensibly UV-opaque may transmit sufficient UV light at the catalyzing wavelength to bond.

The UV-catalyzed bonding method is simple, inexpensive and forms bonds which are optically transparent and may be fully cured in minutes or hours.

1. Use of UV catalyst complexes, such as nanoscale photoactive titania, under sufficient illumination to generate additional hydroxide (OH⁻) and superoxide anion (O₂⁻) or other radicals from in-situ H₂O or other species including organics, that then network with the surfaces and surrounding complexes to generate additional reaction sites.
1.1. Use of pure, highly-distilled water as a source of hydroxyl ions for bonding of many materials, including silicic materials such as fused silica, optical glasses, or other oxides and crystals through surface hydroxyl bonds; this could serve as an adjunct to hydroxyl bonding as set out in claim 1 above.
1.2. Use of acids, such as hydrofluoric acid, or salts that in aqueous solution generate such acids (such as ammonium bifluoride), to bond many materials, including silicic materials such as fused silica, optical glasses to concrete, or other oxides and crystals through in-situ creation of bridging silicate networks or surface hydroxyl bonds; this could serve as an adjunct to hydroxyl bonding as set out in claim 1 above.
1. 2.1. Use of illumination of the UV catalyst before bonding to generate radicals in UV opaque materials.

2. Use of an inorganic photoactive filling mixture on one or both surfaces, such as sodium silicate combined with photoactive tin dioxide, where the filling mixture is capable of forming a network bond between and upon the surfaces.
2.1. Use of an activator to precipitate, chemically cure, gel or set such an inorganic filling mixture for bonding, including laser-damage resistant optical bonding, such that said mixture is significantly less sensitive after gelling to water or hydroxides generated by UV- or other photo-catalysis or to chemical attack of other genesis, than such filling mixtures that gel solely through dehydration.
2.1.1. An example would be bonding silica-based optical glasses with a lithium silicate filling material, using an activator such as sodium bicarbonate or a mineral acid, with or without a photocatalyst such as nanoscale titania. After gelling the bond is significantly stronger, more chemically resistant and less water soluble than one that does not use an activator to set the filling agent, and when a photocatalyst is used, will remain surprisingly photoactive.

3. Designs of processes and inorganic filling mixtures intended to reduce infiltration into porous cementitious or natural surfaces containing various hydroxides, such as but not limited to concrete, mortar and stucco, natural stone, aerogels, xerogels, ceramics, etc., with the use of a photocatalyst so that organics remain, and radicals form, near its surface and do not infiltrate significantly. The mixture is intended to react and fill pores with reaction products, and/or become part of the existing surface, so no binder is required to retain a photocatalyst.
3.1. An example is an aqueous sodium silicate (perhaps 30% solids) mixed with a photocatalyst, such as nanoscale zinc oxide (particle size about 40 nm, perhaps 0.5% by weight), and possibly an acid or salt activator such as sodium fluorosilicate (perhaps 5% by weight), on a concrete substrate (see FIG. 1), under dry ambient conditions.
3.1.1. As is well known, constituents of this mixture, often including orthosilicic acid (written as H₄SiO₄), react with the concrete to fill pores with reaction products (see FIG. 2). This treatment both strengthens the substrate and greatly reduces its permeability.
3.1.2. Observe that the mixture does not serve as a binder for the photocatalyst, since the mixture is consumed while reacting with the alkaline, hydroxide-containing concrete through the well known pozzolanic (acid-base) reaction. This generates high-surface-area calcium silicate hydrate (CSH—often written CaH₂SiO₄), as follows: Ca(OH)₂+H₄SiO₄→CaH₂SiO₄·2 H₂O.
3.1.3. Surprisingly, such high-surface-area reaction products mechanically retain and chemically adsorb, upon and beneath the surface, significant amounts of active photocatalyst. Also, excess unreacted silicic acid components will dehydrate to a vitreous high-surface-area silica gel matrix that fixes photocatalyst and is strongly chemisorbed on the hydroxyl-studded surface. A thin film (<50 μm) of such a material is mechanically, chemically and thermally robust (see FIG. 3). On the other hand if too much mixture is applied, the debris of many unreacted components and reaction products will remain on the surface in a thick film (>500 μm), including salts that are water soluble; these latter will wash off, leaching photocatalyst particles along with them. It is therefore preferable to optimize the volume of mixture applied to the surface in order to chemically saturate the substrate but minimize, and control the chemistry of, any leftover debris film.
3.1.4. In addition to chemisorption, Van der Waals forces strongly affix the smallest nanoparticles, with the highest BET surface areas, to the surface (also FIG. 3). Such small particles are exceedingly difficult to dislodge after dehydration; note for example that optics and electronics cleaning processes require high-energy techniques such as ultrasonics or megasonics to dislodge these physisorbed particles reliably, even from smooth, non-porous surfaces.
3.2. Designs of processes and inorganic filling mixtures as in claim 3 above intended to reduce infiltration into existing porous surfaces, such as concrete, natural stone, etc., through the use of a bonded UV- or other photo-catalyst so that the surface is protected partially or fully from photo-degradation.
3.2.1. Designs of devices utilizing such bonding of surfaces together as set forth in claims 1, 2 and 3 above in such a bonded structure employed in one of the fields selected from optics, electro-optics, micro-optics, microelectronics and microstructure fabrication.
3.2.1.1. One example is a bonded zero-order waveplate made up of two higher-order waveplates whose axes are crossed in order to produce differential delay, of one polarization vs. the other, of some desired fraction of a wavelength at the optical frequency of interest, as is well known in prior art.
3.2.2. Designs utilizing such bonding and surface treatments in one of the fields selected from construction, decoration or maintenance of structures made of concrete, stone, mortar, stucco or other such porous inorganic materials.
